(19) United States
(12) Patent Application Publication
(10) Pub. No.: US 2023/0181384 A1
(43) Pub. Date: Jun. 15, 2023

(54) DIAPER SYSTEM

(71) Applicant: Tabitha Matthews, Granite City, IL (US)

(72) Inventor: Tabitha Matthews, Granite City, IL (US)

(21) Appl. No.: 17/551,447

(22) Filed: Dec. 15, 2021

(51) Int. Cl. A61F 13/49; A61F 13/58

(52) U.S. Cl. CPC A61F 13/49001; A61F 13/49007; A61F 13/58; A61F 2013/51023

(57) ABSTRACT

The present invention provides for and improved diaper system including a proximal structure, a distal structure, and a securing configuration incorporated with the system, whereby each of the proximal structure and distal structure are constructing a range with one or more water absorbent polymers and have therein an interior cavity.

### BACKGROUND OF THE INVENTION

Sanitary devices are continually evolving. It was not very long ago that cloth diapers were the norm and most common in use. As disposable diaper technology advanced, improvements were incorporated. However, sanitary diaper systems still have significant drawbacks relating to comfort and efficacy. There is a need for an improved system addressing these areas. The present invention fulfills this need.

### SUMMARY OF THE INVENTION

The present invention will significantly increase personal hygiene of diaper-wearers by deterring fecal matter from contaminating the genitals and entering the urethra

This invention will significantly decrease the prevalence of urinary tract infections of diaper-wearers caused by a bacteria found is fecal matter, E.Coli and others.

The system of the present invention will decrease the prevalence of Urosepsis of diaper-wearers by significantly decreasing urinary tract infections.

In one embodiment, the system includes:

1. A. proximal or top layer, having:

a) 2 or 5 layer superabsorbent polymer disposable diaper material with waterproof side on the outside which will come in contact with wearers buttocks;

b) an oval shape cut out to allow fecal matter to enter the pouch-dike product; and.

c) Two waterproof adhesive pieces attached to the oval opening with 2 tabs on each piece to allow for better fit, seal and comfort, will be placed onto buttocks surrounding the rectum

A distal or bottom layer—2 or 5 layer superabsorbent polymer disposable diaper material with waterproof side on the outside which will lay within the diaper and have 4 pieces of hook and loop fasteners on it to decrease movement within diaper

3. 2 adhesive pieces configured as follows:

a) waterproof

b) specially designed with. 2 tabs per piece to increase fit and comfort

c) safe for delicate skin

d) will be attached to the oval opening on the top layer

e) the ends of each piece will cross over the other to make a complete seal and allow for personalized fit

f) will attach to the area surrounding the anus

The proximal and distal layers will be the same size and will be connected at the edges

The to edge and the edges of the two sides will then be folded inward to create an accordion-like edge to allow for expansion of the product allowing more holding capacity

In one embodiment the present invention is a diaper system comprising:
a distal structure;
a proximal structure;
an accordion fold structure separating each of said proximal and distal structures;
a single interior cavity formed within each of said proximal and distal structures, wherein said accordion fold structure is constructed and arranged to provide a volume increase as said interior cavity is filled with matter;
a cavity opening on said proximal structure leading to said single interior cavity; and
a securing structure having securing structure opening congruous to said cavity opening and further including releasable adhesive on a proximal surface of said securing structure.

In one embodiment the distal structure and said proximal structure are congruous.

In one embodiment the invention is further including an interior border defining an interior opening.

In one embodiment the invention is further including a substantially circular interior border defining a substantially circular interior opening. In one embodiment the invention is further including an oval interior border defining an oval interior opening.

In one embodiment the distal layer and said proximal layer are formed of one or more water absorbent polymers.

In one embodiment the present invention is a diaper system consisting of:
a distal structure;
a proximal structure;
an accordion fold structure separating each of said proximal and distal structures;
a single interior cavity formed within each of said proximal and distal structures, wherein said accordion fold structure is constructed and arranged to provide a volume increase as said interior cavity is filled with matter;
a cavity opening on said proximal structure leading to said single interior cavity; and

a securing structure having securing structure opening congruous to said cavity opening and further including releasable adhesive on a proximal surface of said securing structure, wherein the distal structure and said proximal structure are congruous and include any one or more of the limitations and structures provided herein.

### BRIEF DESCRIPTION OF THE SEVERAL VIEWS OF THE DRAWINGS

FIG. 1 is a front plan view of the proximal pad according to one embodiment of the present invention.

FIG. 2 is a top perspective you according to one embodiment of the present invention.

FIG. 3 is a top side view according to one embodiment of the present invention.

FIG. 4 is a right side view according to one embodiment of the present invention.

FIG. 5 is an exploded view demonstrating components according to one embodiment of the present invention.

FIG. 6 is a right side cross section view along section lines 6-6 from FIG. 1.

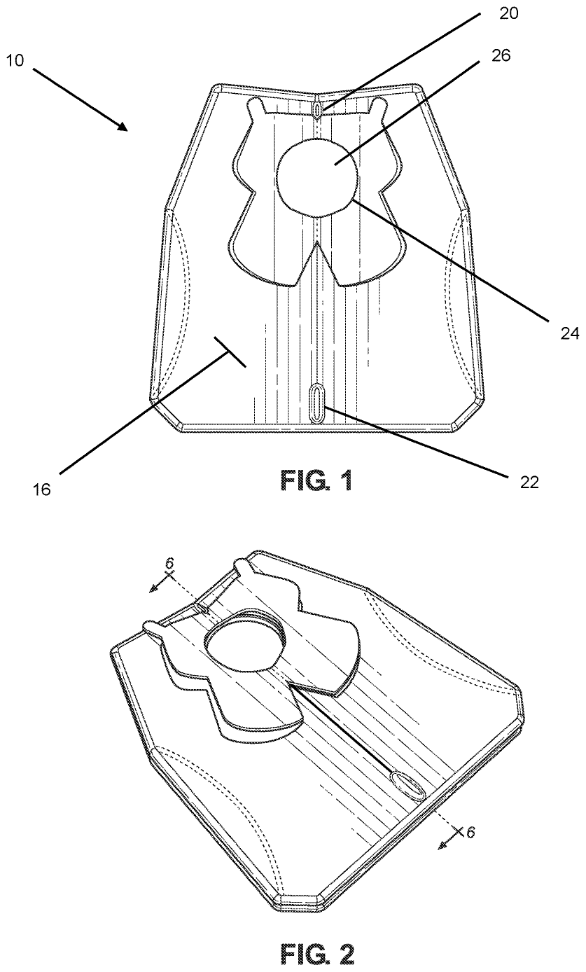

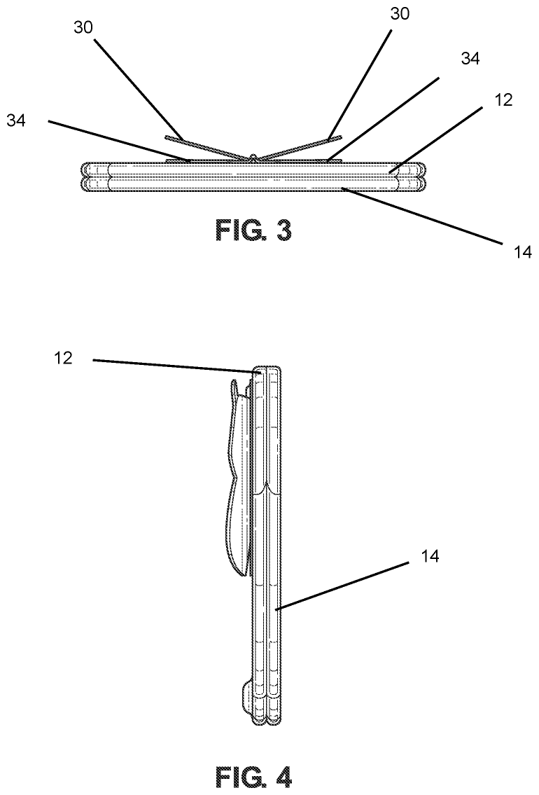

### DETAILED DESCRIPTION OF THE PREFERRED EMBODIMENTS

Diaper system 10 is formed with proximal structure 12 and distal structure 14. As used herein, proximal will refer to those directions intended to be closer to the person wearing the system of the present invention and distal are those directions away from the wearer. Proximal structure 12 has proximal surface 16. Distal structure 14 has distal surface 18. Each of the proximal and distal structures as described herein and understood by the figures form a single contiguous structure with an interior cavity for collecting waste matter. These structures together form a storage configuration for system 10. They are two portions of a single system. Single interior cavity 17 is formed on the interior and encompasses the interior of both proximal structure 12 and distal structure 14. The Single interior cavity 17 is configured as being provided in a first compact configuration that expands along accordion fold 19 to a second or expanded configuration when matter is introduced into single interior cavity 17 through cavity access opening 24. In use, system 10 has single interior cavity floor 15 on the interior of distal structure 14.

Incorporated onto proximal surface 16 is first tab 20 and second tab 22. Each of first tab 20 and second tab 22 are configured as grab tab devices were by a user can grasp and position system 10 as needed. System 10 includes border 24 that defines system cavity access 26.

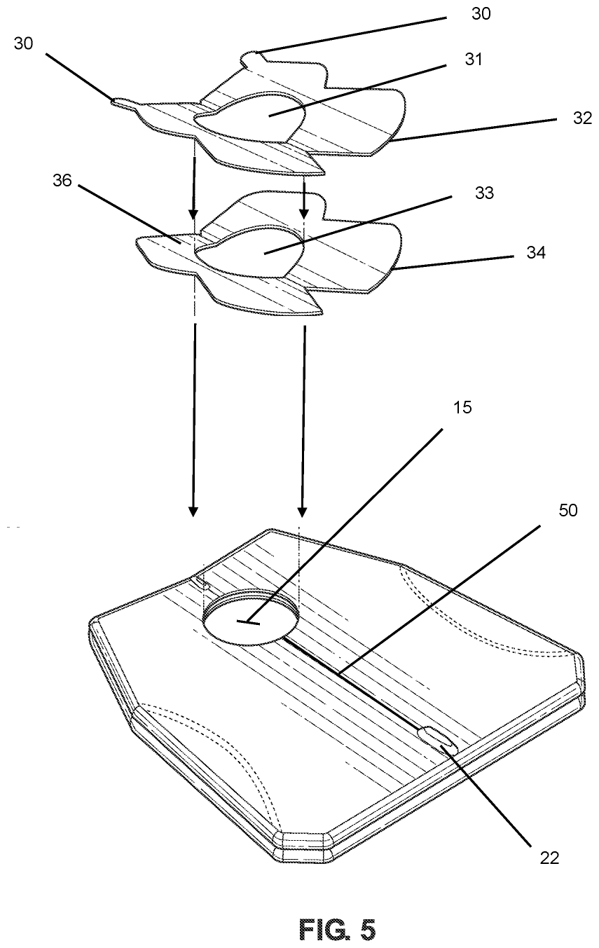

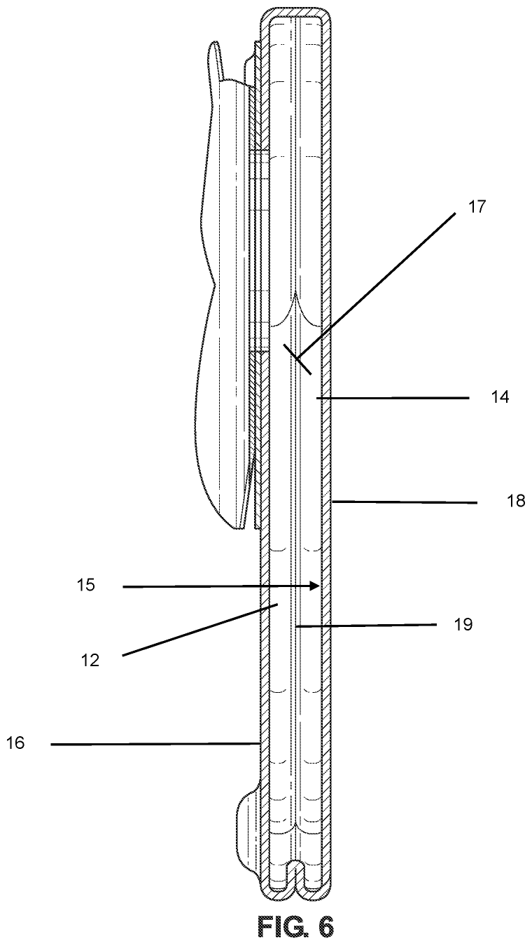

In one embodiment, as demonstrated in FIG. 5, a proximal removable adhesive backing layer 32 is constructed with a pair of peel tabs 30 such that a user will remove adhesive backing layer 32 and expose adhesive 36 disposed on the proximal surface of securing structure 34. As demonstrated in the figures, securing structure 34 has cavity 33 congruous

The system further includes central fold 50 as indicated on the figures.

In use, a user will remove adhesive backing 30 to utilizing pull tabs 30. Adhesive 36 is exposed and used to secure system 10 onto the skin of a user.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

What is claimed:

1. A diaper system comprising:
a distal structure;
a proximal structure;
an accordion fold structure separating each of said proximal and distal structures;
a single interior cavity formed within each of said proximal and distal structures, wherein said accordion fold structure is constructed and arranged to provide a volume increase as said interior cavity is filled with matter;
a cavity opening on said proximal structure leading to said single interior cavity; and
a securing structure having securing structure opening congruous to said cavity opening and further including releasable adhesive on a proximal surface of said securing structure.

2. The diaper system of claim 1 wherein said distal structure and said proximal structure are congruous.

3. The diaper system of claim 1 further including an interior border defining an interior opening.

4. The diaper system of claim 1 further including a substantially circular interior border defining a substantially circular interior opening.

5. The diaper system of claim 1 further including an oval interior border defining an oval interior opening.

6. The diaper system of claim 1 wherein said distal layer and said proximal layer are formed of one or more water absorbent polymers.
